(19) United States
(12) Patent Application Publication
(10) Pub. No.: US 2025/0312295 A1
(43) Pub. Date: Oct. 9, 2025

(54) SUBLINGUAL SUBSTANCE ABUSE DISORDER AND ANTI-ADDICTION LOZENGE

(71) Applicant: Synergistic Therapeutics, LLC, Naples, FL (US)

(72) Inventors: Anthony H. Salce, JR., Naples, FL (US); Michael A. Aquino, Naples, FL (US)

(21) Appl. No.: 19/170,435

(22) Filed: Apr. 4, 2025

Related U.S. Application Data
(60) Provisional application No. 63/575,109, filed on Apr. 5, 2024.

(51) Int. Cl. A61K 31/137; A61K 9/00; A61K 31/485; A61K 47/02; A61K 47/10; A61K 47/12; A61K 47/46

(52) U.S. Cl. CPC A61K 31/137; A61K 9/0056; A61K 31/485; A61K 47/02; A61K 47/10; A61K 47/12; A61K 47/46

(57) ABSTRACT

Formulations and methods are described to produce sublingual substance abuse disorder and anti-addiction lozenges. The lozenges may include troche base, ketamine, and buprenorphine. The lozenges may include 0.25 weight percent to 2.53 weight percent ketamine and 0.61 weight percent to 4.88 weight percent buprenorphine. The methods may include placing troche base into a chamber and applying heat to the chamber to melt the troche base. The methods may include adding a first ingredient into the chamber and mixing the first ingredient into the melted troche base. The first ingredient may include ketamine. The methods may include adding a second ingredient into the chamber and mixing the second ingredient into the melted troche base with the first ingredient. The second ingredient may include buprenorphine. The methods may include pouring the melted mixture into a mold. The methods may include cooling the melted mixture in the mold to form the lozenge.

### FIELD OF INVENTION

The present application relates generally to treatment for substance abuse disorder and addiction including opioid abuse disorder and addiction and alcohol abuse disorder and addiction.

### BACKGROUND

From a neurobiological point of view, opioids may play a crucial role in pain processing, stress responses, respiration, gastrointestinal transit, and the endocrine system, in particular, the hypothalamus-pituitary-adrenal gland (HPA) axis, and immune functions, and their dysregulation exerts an important role in attachment, loss, anhedonia, and MDD itself. The endogenous opioid system may be comprised of three different G-protein coupled receptors (GPCRs), μ-, δ-, and κ-opioids receptors (MORs, DORs, and KORs, respectively) that are linked with a family of endogenous opioid peptides known as β-endorphin, enkephalins, and dynorphins. These receptors may be widespread in human central and peripheral nervous systems, with a high density in limbic areas that may explain their role in reward processing and mood control and may support their use to treat emotional dysfunction. These pharmacological options may modulate BDNF activity and enhance neurogenesis in the hippocampus.

MOR activation in the dorsal raphe nucleus (DRN) and ventral tegmental area (VTA) by local GABAergic interneurons may disinhibit both 5-HT and DA neurons but inhibit noradrenergic neurons. The MOR-mediated mechanism of mood control may be more complex than described above; in fact, some studies show a paradoxical depressive-like potential of MOR according to the evidence that two groups of MOR KO mice appeared to have decreased anxiety and depressive-like behaviors. DOR that is antagonized by buprenorphine together with encephalin, may have a mood-enhancing activity, but it is not clear how it regulates the reward process. KOR may also be antagonized by buprenorphine and exhibit a major anti-reward role and be able to reduce reward tonically. This type of activity may be potentiated by different stressors and may play a role in various stress-induced psychopathological conditions. The dynorphin/KOR system, through the action on DA neurons in the nucleus accumbens (NAc), may be linked to depressive-like behaviors.

Buprenorphine may undergo hepatic metabolism primarily by CYP450-3A4 and CYP 2C8 and, after N-dealkylation, may be transformed in nor-buprenorphine. These two compounds may be glucuronide and later excreted by the renal and biliary route. About 70% of buprenorphine may be fecally excreted, however, some buprenorphine may be re-absorbed as free buprenorphine, and nor-buprenorphine.

### SUMMARY

In some examples lozenges are described. The lozenges may include troche base, ketamine, and buprenorphine. The lozenges may include 0.25 weight percent to 2.53 weight percent ketamine and 0.61 weight percent to 4.88 weight percent buprenorphine.

In aspects the lozenges further include 0.79 weight percent to 2.03 weight percent silica gel powder.

In aspects the lozenges further include 0.09 weight percent to 2.33 weight percent citric acid powder.

In aspects, the lozenges further include 1.29 weight percent to 3.15 weight percent acacia powder.

In aspects, the lozenges further include a liquid flavoring.

In aspects, the troche base includes a blend of polyethylene glycols.

In aspects, the ketamine includes ketamine hydrochloride powder.

In some examples, methods to produce lozenges are described. The methods may include placing troche base into a chamber and applying heat to the chamber to melt the troche base. The methods may include adding a first ingredient into the chamber and mixing the first ingredient into the melted troche base. The first ingredient may include ketamine. The methods may include adding a second ingredient into the chamber and mixing the second ingredient into the melted troche base with the first ingredient. The second ingredient may include buprenorphine. The methods may include pouring the melted mixture into a mold. The methods may include cooling the melted mixture in the mold to form the lozenge.

In aspects, the mold includes thirty uniformly sized cavities.

In some examples lozenges are described. The lozenges may include 0.25 weight percent to 2.53 weight percent ketamine, 0.61 weight percent to 4.88 weight percent buprenorphine, 0.79 weight percent to 2.03 weight percent silica gel powder, 0.09 weight percent to 2.33 weight percent citric acid powder, 1.29 weight percent to 3.15 weight percent acacia powder, and a troche base.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example system that can be utilized to produce a sublingual substance abuse disorder and anti-addiction lozenge; and

FIG. 2 illustrates a flow diagram of an example process to produce a sublingual substance abuse disorder and anti-addiction lozenge;

all arranged according to at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

It will be understood that any compound, material or substance which is expressly or implicitly disclosed in the specification and/or recited in a claim as belonging to a group or structurally, compositionally and/or functionally related compounds, materials or substances, includes individual representatives of the group and all combinations thereof.

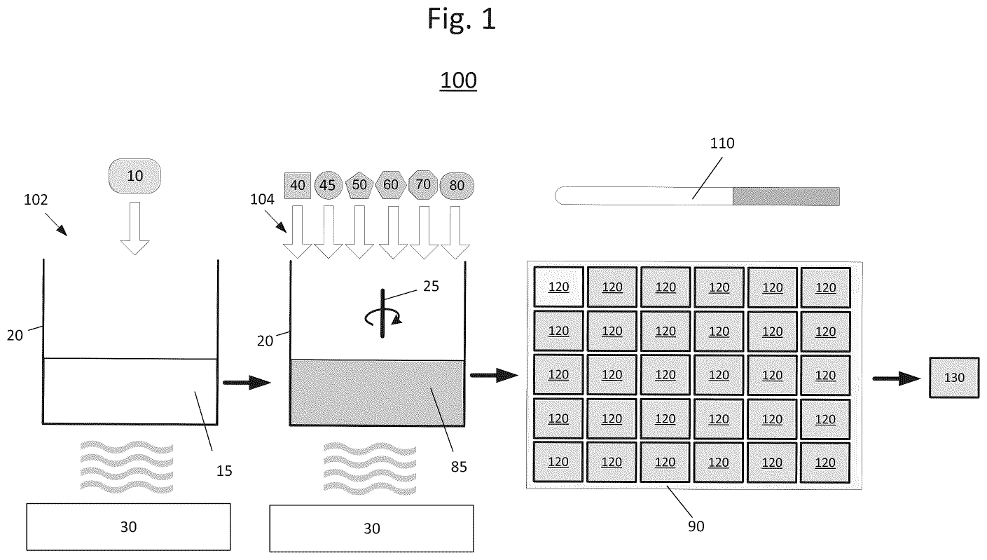

FIG. 1 illustrates an example system that can be utilized to produce a sublingual substance abuse disorder and anti-addiction lozenge, arranged in accordance with at least some embodiments presented herein. As discussed in more detail below, a sublingual substance abuse disorder and anti-addiction lozenge may be effective in the treatment of depression and anxiety.

System 100 may include a chamber 20, a heater 30 and a lozenge mold 90. At 102 a troche base 10 may be placed in within chamber 20 and melted by heat from heater 30 to produce melted troche base 15. Troche base 10 may be a sweetened blend of polyethylene glycols (PEGs) used to compound medicinal lozenges or troches. Troche base 10 may be white and/or translucent in appearance and be in the shape of small pellet pieces at room temperature. Troche base 10 may be solid at room temperatures of 20 to 25 degrees Celsius. Heater 30 may supply heat to increase a temperature of troche base 10 to about 45 to 51 degrees Celsius and melt troche base 10 to produce melted troche base 15.

At 104, a mixing instrument 25 may be inserted into chamber 20 and ingredients 40, 45, 50, 60, 70, and 80 may each be individually and respectively added and blended into melted troche base 15. Mixing instrument 25 may be a manual mixing instrument such as a spoon or whisk, or an automated mixer.

Ingredient 40 may be in powder form. Ingredient 40 may include ketamine. Ingredient 40 may include ketamine hydrochloride (HCl) powder. Ingredient 40 may include ketamine hydrochloride as a water-soluble, white crystalline powder with a pKa of 7.5. Ingredient 40 may have a free base of ketamine with a lipid solubility 10 times that of thiopentone. Ingredient 40 may be a commercially available pharmaceutical form of ketamine hydrochloride as an aqueous solution for injection of the racemic mixture of the hydrochloride salt. Ingredient 40 may be a dissociative anesthetic and the dissociation component may refer to a functional and electrophysiological dissociation of thalamo neocortical and limbic systems. Ingredient 40 may induce a feeling of dissociation of the mind from the body. Ingredient 40 may be prescribed to treat depression and anxiety and other categories of depression such as major depressive disorder, treatment resistant depression, postpartum, post-traumatic stress disorder, bipolar disorder and addiction.

Ingredient 45 may be in powder form. Ingredient 45 may be a synthetic partial opioid agonist analgesic derived from thebaine. Ingredient 45 may be buprenorphine. Ingredient 45 may be a white, crystalline powder that is soluble 17 mg/mL in water and 42 mg/mL in alcohol at room temperature.

Ingredient 50 may be in powder form. Ingredient 50 may include silica gel powder. Ingredient 50 may be granular, vitreous in appearance, and porous. Ingredient 50 may be tough and hard in texture and be more solid than a gelatin or agar. Ingredient 50 may be a naturally occurring mineral that is purified and processed into a granular form. Ingredient 50 may include a strong affinity for water molecules. Ingredient 50 may be silicon dioxide produced synthetically from sodium silicate. Ingredient 50 may have an average pore size of about 2.4 nanometers. Ingredient 50 may be a suspending agent and may keep materials from settling at the bottom of a mold cavity during cooling.

Ingredient 60 may be in powder form. Ingredient 60 may include a weak organic tribasic acid. Ingredient 60 may include citrate. Ingredient 60 may include citric acid powder. Ingredient 60 may include an acidifier, a flavoring, a chelating agent, or a pH adjusting agent. Ingredient 60 may include a processing aid.

Ingredient 70 may be in powder form. Ingredient 70 may include acacia powder. Ingredient 70 may include gum exuded from the acacia tree. Ingredient 70 may include dietary fiber that can dissolve in water. Ingredient 70 may add texture and smoothness to a sublingual substance abuse disorder and anti-addiction lozenge.

Ingredient 80 may be in liquid form. Ingredient 80 may be a liquid flavoring. Ingredient 80 may include a liquid confection product. Ingredient 80 may enhance digestion and taste of a sublingual anti-opioid addiction lozenge. Ingredient 80 may include mint, cherry, orange, lime, lemon or grape flavors.

As shown at 104, ingredient 40 may be added to chamber 20 and blended into melted troche base 15. Ingredient 40 may be geometrically diluted into melted troche base 15. Ingredient 40 may be mixed until ingredient 40 is evenly distributed throughout melted troche base 15 as indicated by an even distribution of a color of ingredient 40 throughout melted troche base 15.

As shown at 104, ingredient 45 may be added to chamber 20 and blended into melted troche base 15. Ingredient 45 may be geometrically diluted into melted troche base 15. Ingredient 45 may be mixed until ingredient 40 is evenly distributed throughout melted troche base 15 as indicated by an even distribution of a color of ingredient 45 throughout melted troche base 15.

As shown at 104, ingredient 50 may be added to chamber 20 and blended into melted troche base 15. Ingredient 50 may be geometrically diluted into melted troche base 15. Ingredient 50 may be mixed until ingredient 50 is evenly distributed throughout melted troche base 15 as indicated by an even distribution of a color of ingredient 50 throughout melted troche base 15.

As shown at 104, ingredient 60 may be added to chamber 20 and blended into melted troche base 15. Ingredient 60 may be geometrically diluted into melted troche base 15. Ingredient 60 may be mixed until ingredient 60 is evenly distributed throughout melted troche base 15 as indicated by an even distribution of a color of ingredient 60 throughout melted troche base 15.

As shown at 104, ingredient 70 may be added to chamber 20 and blended into melted troche base 15. Ingredient 70 may be geometrically diluted into melted troche base 15. Ingredient 70 may be mixed until ingredient 70 is evenly distributed throughout melted troche base 15 as indicated by an even distribution of a color of ingredient 70 throughout melted troche base 15.

As shown at 104, ingredient 80 may be added to chamber 20 and blended into melted troche base 15. Ingredient 80 may be mixed until ingredient 80 is evenly distributed throughout melted troche base 15 as indicated by an even distribution of a color of ingredient 80 throughout melted troche base 15.

A melted lozenge mixture 85 may be formed by mixing ingredients 40, 45, 50, 60 70, and 80 into melted troche base 15. Melted lozenge mixture 85 may be poured into cavities 120 of lozenge mold 90. Lozenge mold 90 may be plastic, anodized aluminum, or some other non-permeable material, and may be configured to form equal sized lozenges. Lozenge mold 90 may include 30 uniformly sized cavities 120. Melted lozenge mixture 85 may be poured into cavities 120 of lozenge mold 90 so as to completely fill cavities 120. A scrapper or spatula 110 may be used to level and even out poured melted lozenge mixture 85 in cavities 120 of lozenge mold 90. Spatula 110 may also be used to wipe any excess melted lozenge mixture 85 off of lozenge mold 90.

Lozenge mold 90, with cavities 120 filled with melted lozenge mixture 85, may be cooled to room temperature of 20 to 25 degrees Celsius to form lozenge 130. Lozenge 130 may be a solid lozenge with ingredients 40, 45, 50, 60, 70, and 80 distributed evenly throughout lozenge 130. Lozenge 130 may weigh approximately 100-200 mg. Lozenge 130 may include about 0.25 weight percent to about 2.53 weight percent of ingredient 40 which may be equivalent to a minimum of 2.5 mg and a maximum of 25 mg of ingredient 40. Lozenge 130 may include about 0.61 weight percent to about 4.88 weight percent of ingredient 45 which may be equivalent to a minimum of 2.0 mg to a maximum of 8.0 mg of ingredient 45. Lozenge 130 may include about 0.79 weight percent to about 2.03 weight percent of ingredient 50. Lozenge 130 may include about 0.09 weight percent to about 2.33 weight percent of ingredient 60. Lozenge 130 may include about 1.29 weight percent to about 3.15 weight percent of ingredient 70.

An example lozenge 130 may be formed from the following quantities:

0.0025 to 0.025 grams of ingredient 40. Ingredient 40 may be ketamine HCl powder.

0.0020 to 0.0080 grams of ingredient 45. Ingredient 45 may be buprenorphine powder.

About 0.041 grams of ingredient 50. Ingredient 50 may be silica gel powder.

About 0.021 grams of ingredient 60. Ingredient 60 may be citric acid powder.

About 0.022 grams of ingredient 70. Ingredient 70 may be acacia powder.

About 0.995 grams of troche base 10.

About 0.200 ml of ingredient 80. Ingredient 80 may be tutti frutti flavor liquid.

An example batch to produce 30 lozenges 130 may be formed from the following quantities:

0.075 to 0.75 grams of ingredient 40. Ingredient 40 may be ketamine HCl powder.

0.06 to 0.24 grams of ingredient 45. Ingredient 45 may be buprenorphine powder.

About 1.23 grams of ingredient 50. Ingredient 50 may be silica gel powder.

About 0.63 grams of ingredient 60. Ingredient 60 may be citric acid powder.

About 0.67 grams of ingredient 70. Ingredient 70 may be acacia powder.

About 29.85 grams of troche base 10.

About 6.0 ml of ingredient 80. Ingredient 80 may be tutti frutti flavor liquid.

In an example, lozenge 130 may be formulated to include 10 mg of ingredient 40, ketamine HCl, and 5 mg of ingredient 45, buprenorphine powder, which may be an amount of active ingredients to be administered to a patient depending on weight once or twice daily.

It was unexpectedly found that when ingredient 40, ketamine, was utilized with ingredient 45, buprenorphine, the combined medication possessed outstanding results in treating the severance of opioid addiction and alcohol addiction, and enhanced treatment for the abuse disorder of these substances. Ingredient 40, ketamine in low doses as described above, may enhance anti-addition effects of ingredient 45, buprenorphine and a combination of these ingredients may provide superior treatment in the severance of opioid addiction and alcohol addiction. As with all medications used in Medication-Assisted Treatment (MAT), buprenorphine should be prescribed as part of a comprehensive treatment plan that may not initially include counseling. Micro dosing therapies may provide patients with effective results for utilizing a daily dosage. Ketamine and buprenorphine may be the first medications to treat opioid use disorder (OUD) which may be prescribed or dispensed in physician offices and may significantly increase access to treatment. Ketamine and buprenorphine may also be prescribed to treat moderate to severe pain and may be prescribed to treat depression and anxiety and other categories of depression such as major depressive disorder, treatment resistant depression, postpartum, post-traumatic stress disorder, bipolar disorder and addiction. For the purposes of opioid treatment in most cases a patient may realize the effects of the medication and be completely severed from opioids within 4-6 weeks. Ketamine and buprenorphine may also be prescribed to treat alcohol abuse disorder and alcohol addiction.

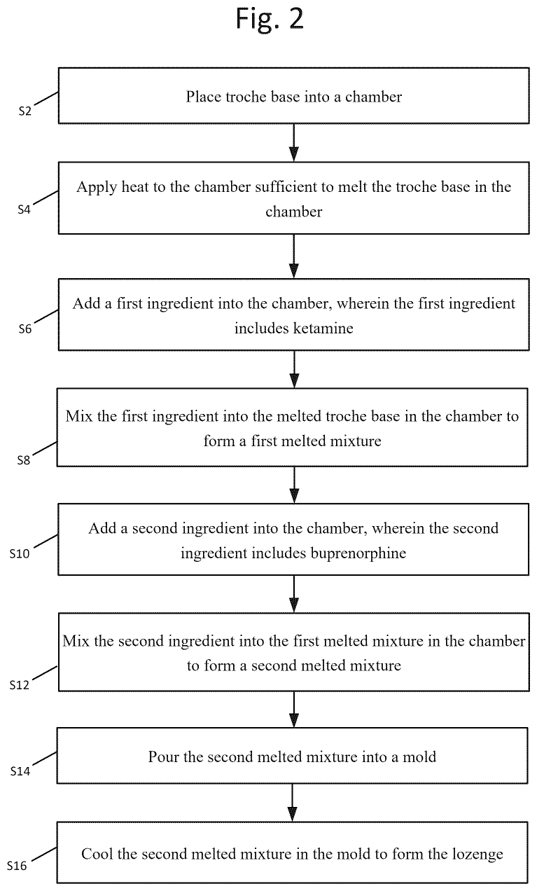

FIG. 2 illustrates a flow diagram of an example process to produce a sublingual substance abuse disorder and anti-addiction lozenge 130. The process in FIG. 2 could be implemented using, for example, system 100 discussed above. An example process may include one or more operations, actions, or functions as illustrated by one or more of blocks S2, S4, S6, S8, S10, S12, and/or S14. Although illustrated as discrete blocks, various blocks may be divided into additional blocks, combined into fewer blocks, or eliminated, depending on the desired implementation.

Processing may begin at block S2, “Place troche base into a chamber.” At block S2, a troche base is placed into a chamber. The troche base includes a blend of polyethylene glycols (PEGs). The troche base is white and/or translucent in appearance and in the shape of small pellet pieces. The troche base is solid at room temperatures of 20 to 25 degrees Celsius.

Processing may continue from block S2 to block S4, “Apply heat to the chamber sufficient to melt the troche base in the chamber.” At block S4, heat is applied to the chamber sufficient to melt the troche base. Heat is applied to the chamber sufficient to increase a temperature of the troche base to about 45 to 51 degrees Celsius and melt the troche base.

Processing may continue from block S4 to block S6, “Add a first ingredient into the chamber, wherein the first ingredient includes ketamine.” At block S6, a first ingredient is added to the chamber. The first ingredient includes ketamine. The first ingredient includes ketamine hydrochloride (HCl) powder.

Processing may continue from block S6 to block S8, “Mix the first ingredient into the melted troche base in the chamber to form a first melted mixture.” At block S8, the first ingredient is mixed into the melted troche base in the chamber. The mixing is performed by a manual mixing instrument such as a spoon or whisk, or an automated mixer. The first ingredient is mixed until the first ingredient is evenly distributed throughout the melted troche base as indicated by an even distribution of a color of the first ingredient throughout the melted troche base.

Processing may continue from block S8 to block S10, “Add a second ingredient into the chamber, wherein the second ingredient includes buprenorphine.” At block S10, a second ingredient is added to the chamber. The second ingredient includes buprenorphine. The first ingredient may include ketamine hydrochloride (HCl) powder.

Processing may continue from block S10 to block S12, “Mix the second ingredient into the first melted mixture in the chamber to form a second melted mixture.” At block S12, the second ingredient is mixed into the first melted mixture in the chamber. The mixing is performed by a manual mixing instrument such as a spoon or whisk, or an automated mixer. The second ingredient is mixed until the second ingredient is evenly distributed throughout the first melted mixture as indicated by an even distribution of a color of the second ingredient throughout the first melted mixture.

Processing may continue from block S12 to block S14, “Pour the second melted mixture into a mold.” At block S14, the second melted mixture is poured into a mold. The mold is plastic, anodized aluminum, or some other non-permeable material. The mold is configured with cavities to form uniform sized lozenges. The second melted mixture is poured into the cavities of the mold so as to completely fill the cavities of the mold. A scrapper or spatula is used to level and even out poured second melted mixture in the cavities of the mold. The spatula is also used to wipe any excess second melted mixture.

Processing may continue from block S14 to block S16, “Cool the second melted mixture in the mold to form the lozenge.” At block S16, the second melted mixture in the mold is cooled to form the lozenge. The second melted mixture is cooled to room temperature of 20 to 25 degrees Celsius.

A system in accordance with the present disclosure may be effective to produce a sublingual substance abuse disorder and anti-addiction lozenge. A potential benefit of the present application may be the treatment of opioid abuse disorder and opioid addiction. An embodiment of the present application may provide rapid resolution for opioid addiction and a patient may be completely severed from opioids within 4-6 weeks. A potential benefit of the present application may be the treatment of alcohol abuse disorder and alcohol addiction.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A lozenge comprising:
a troche base;
0.25 weight percent to 2.53 weight percent ketamine; and
0.61 weight percent to 4.88 weight percent buprenorphine;
wherein the lozenge is a treatment for substance abuse disorder and addiction.

2. The lozenge of claim 1, further comprising 0.79 weight percent to 2.03 weight percent silica gel powder.

3. The lozenge of claim 1, further comprising 0.09 weight percent to 2.33 weight percent citric acid powder.

4. The lozenge of claim 1, further comprising 1.29 weight percent to 3.15 weight percent acacia powder.

5. The lozenge of claim 1, further comprising:
0.79 weight percent to 2.03 weight percent silica gel powder;
0.09 weight percent to 2.33 weight percent citric acid powder; and
1.29 weight percent to 3.15 weight percent acacia powder.

6. The lozenge of claim 5, further comprising a liquid flavoring.

7. The lozenge of claim 1, wherein the troche base includes a blend of polyethylene glycols.

8. The lozenge of claim 1, wherein the ketamine includes ketamine hydrochloride powder.

9. A method to produce a lozenge, the method comprising:
placing troche base into a chamber;
applying heat to the chamber sufficient to melt the troche base in the chamber;
adding a first ingredient into the chamber, wherein the first ingredient includes ketamine;
mixing the first ingredient into the melted troche base in the chamber to form a first melted mixture;
adding a second ingredient into the chamber, wherein the second ingredient includes buprenorphine;
mixing the second ingredient into the first melted mixture in the chamber to form a second melted mixture;
pouring the second melted mixture into a mold; and
cooling the second melted mixture in the mold to form the lozenge;
wherein the lozenge is a treatment for substance abuse disorder and addiction.

10. The method of claim 9, further comprising, prior to pouring the second melted mixture into the mold:
adding a third ingredient to the chamber, wherein the third ingredient includes silica gel powder; and
mixing the third ingredient into the second melted mixture.

11. The method of claim 9, further comprising, prior to pouring the melted mixture into the mold:
adding a third ingredient to the, wherein the third ingredient includes citric acid powder; and
mixing the third ingredient into the second melted mixture.

12. The method of claim 9, further comprising, prior to pouring the melted mixture into the mold:
adding a third ingredient to the chamber, wherein the third ingredient includes acacia powder; and
mixing the third ingredient into the second melted mixture.

13. The method of claim 9, further comprising, prior to pouring the melted mixture into the mold:
adding a third ingredient to the chamber, wherein the third ingredient includes silica gel powder;
mixing the third ingredient into the second melted mixture;
adding a fourth ingredient to the chamber, wherein the four ingredient includes citric acid powder;
mixing the fourth ingredient into the second melted mixture.;
adding a fifth ingredient to the chamber, wherein the fifth ingredient includes acacia powder; and
mixing the fifth ingredient into the second melted mixture.

14. The method of claim 13, further comprising, prior to pouring the fifth melted mixture into the mold:
adding a liquid flavoring to the chamber; and
mixing the liquid flavoring into the second melted mixture.

15. The method of claim 13, wherein the mold includes thirty uniformly sized cavities.

16. The method of claim 9, wherein the ketamine includes ketamine hydrochloride powder.

17. A troche, lozenge comprising:
0.25 weight percent to 2.53 weight percent ketamine;
0.61 weight percent to 4.88 weight percent buprenorphine;
0.79 weight percent to 2.03 weight percent silica gel powder;
0.09 weight percent to 2.33 weight percent citric acid powder;
1.29 weight percent to 3.15 weight percent acacia powder; and
a troche base;
wherein the lozenge is a treatment for substance abuse disorder and addiction.

18. The lozenge of claim 17, wherein the troche base includes a blend of polyethylene glycols.

19. The lozenge of claim 17, wherein the ketamine includes ketamine hydrochloride powder.

20. The lozenge of claim 17, further comprising a liquid flavoring
